(19) United States
(12) Patent Application Publication
(10) Pub. No.: US 2017/0347952 A1
(43) Pub. Date: Dec. 7, 2017

(54) SENSORS FOR ORAL DOSAGE FORMS

(71) Applicant: RAINBOW MEDICAL LTD., Herzliya (IL)

(72) Inventor: Yossi GROSS, Moshav Mazor (IL)

(73) Assignee: RAINBOW MEDICAL LTD., Herzliya (IL)

(21) Appl. No.: 15/174,144

(22) Filed: Jun. 6, 2016

(51) Int. Cl. A61B 5/00; A61J 3/00; A61K 9/48

(52) U.S. Cl. CPC A61B 5/4833; A61K 9/4833; A61K 9/4825; A61B 5/0002; A61K 9/4808; A61J 3/007; A61J 2200/30

(57) ABSTRACT

Apparatus is provided that includes a drug capsule containing an oral drug, and a sensing apparatus that includes a housing and a sensor. The housing is shaped so as to define exactly one hemispherical portion and exactly one cylindrical portion, which together define an internal surface tightly fitted to at least a portion of an external surface of the drug capsule. The sensor includes first and second electrodes, which comprise first and second electrode surfaces, respectively; and circuitry, which (a) is attached to the housing, (b) is electrically coupled to the first and the second electrode surfaces, and (c) is configured to drive a current between the first and the second electrode surfaces. Other embodiments are also described.

### FIELD OF THE APPLICATION

The present invention relates generally to electronic sensors, and specifically to ingestible sensors for monitoring drug compliance.

### BACKGROUND OF THE APPLICATION

Poor medication adherence is a common problem that leads to increased morbidity and death and is estimated to incur costs of approximately $100 billion per year (Osterberg L et al., “Adherence to medication,” N Engl J Med. 2005 Aug. 4; 353 ( 5) :487-97). Technological solutions to improve medication adherence have been proposed, including attaching radio frequency identification (RFID) tags and antennas to drug pills.

PCT Publication WO 2009/042812 to Hafezi et al. describes virtual dipole signal amplification for in-body devices, such as implantable and ingestible devices. Aspects of the in-body deployable antennas of the invention include antennas configured to go from a first configuration to a second configuration following placement in a living body, e.g., via ingestion or implantation. Embodiments of the in-body devices are configured to emit a detectable signal upon contact with a target physiological site. Also provided are methods of making and using the devices of the invention.

### SUMMARY OF THE APPLICATION

In some embodiments of the present invention, a sensing apparatus is provided for use with an oral dosage form containing an oral drug. The sensing apparatus is configured emit a detectable signal upon contact with a target physiological liquid inside a body of a human subject, such as gastric acid, after the oral dosage form has been swallowed with the sensing apparatus attached thereto. The signal is detectable by a separate sensing unit, which is typically configured to be disposed external the subject's body.

The sensing apparatus is typically used to measure patient drug compliance, by definitely confirming that the patient has swallowed a particular oral dosage form as directed by a physician, as well as by creating a record of the precise times of drug administration. Accurate confirmation of patient drug compliance is important both for medical outcomes in individual patients, as well as for ensuring accurate results in multi-patient clinical drug trials (in which case some of the oral dosage forms may be placebos). For some applications, the emitted detectable signal is a generic signal that indicates that the oral dosage form has been swallowed (and reached a target site, such as the stomach or intestine). Alternatively, the signal may include a unique signature for a particular pill or batch of pills.

In some embodiments of the present invention, the sensing apparatus is shaped so as to define a surface that is attached to at least a portion of an external surface of the oral dosage form by friction.

In some embodiments of the present invention, the oral dosage form is a drug capsule containing the oral drug. The sensing apparatus comprises a housing, which is shaped so as to define exactly one hemispherical portion and exactly one cylindrical portion, which together define an internal surface for tight fitting to at least a portion of an external surface of the drug capsule. The sensing apparatus further comprises a sensor, which comprises:

first and second electrodes, which comprise first and second electrode surfaces, respectively; and

circuitry, which (a) is attached to the housing, (b) is electrically coupled to the first and the second electrode surfaces, and (c) is configured to drive a current between the first and the second electrode surfaces.

The driving of the current between the first and the second electrode surfaces emits a detectable signal.

For some applications, the sensor (and the sensing apparatus) does not comprise a complete battery. Instead, the sensor derives energy from an acid (such as gastric acid) when the sensor comes in contact with the acid, such as when the sensing apparatus is disposed in the stomach upon being swallowed. Thus, contact with the acid (such as gastric acid) activates the circuitry.

In some embodiments of the present invention, the oral dosage form is a disk-shaped drug tablet that comprises the oral drug. The drug tablet has two major opposing surfaces connected by a cylindrical side wall. The sensing apparatus comprises a housing, which is shaped so as to define a ring for tight fitting around an external surface of the cylindrical side wall of the drug tablet. The sensing apparatus further comprises a sensor, as described above.

In some embodiments of the present invention, a sensing apparatus comprises a sensor, which is configured to assume compressed and expanded configurations. The sensor comprises (a) first and second electrodes, which comprise first and second electrode surfaces, respectively, and (b) circuitry, which is electrically coupled to the first and the second electrode surfaces. The sensor is:

configured such that when the sensor is in the compressed configuration, the first and the second electrode surfaces are disposed at a closest compressed-configuration distance from each other,

configured such that when the sensor is in the expanded configuration, first and second electrode surfaces are disposed at a closest expanded-configuration distance from each other, the closest expanded-configuration distance equal to between 2 and 8 times the closest compressed-configuration distance, and

configured to drive a current between the first and the second electrode surfaces when the sensor is in the expanded configuration.

The driving of the current between the first and the second electrode surfaces emits a detectable signal, such as described above. For some applications, the sensor comprises a hydrogel, which is configured to undergo expansion upon contact with a liquid (e.g., having a pH of 3 (such as gastric acid)), thereby transitioning the sensor from the compressed configuration to the expanded configuration, and increasing a closest distance between the first and the second electrode surfaces from the closest compressed-configuration distance to the closest expanded-configuration distance. For some applications, the first and the second electrodes further comprise first and second elongate support structures, respectively.

In some embodiments of the present invention, a method of assembling or manufacturing is provided, which comprises providing any of the sensing apparatus and oral dosage forms described herein, holding the oral dosage form (e.g., by an element of a manufacturing system, such as by a robot, or by a human hand), and attaching the sensing apparatus to the oral dosage form.

There is therefore provided, in accordance with an application of the present invention, apparatus including:

an oral drug;

a drug capsule containing the oral drug; and

a sensing apparatus, which includes:
a housing, which is shaped so as to define exactly one hemispherical portion and exactly one cylindrical portion, which together define an internal surface tightly fitted to at least a portion of an external surface of the drug capsule; and
a sensor, which includes:
   first and second electrodes, which include first and second electrode surfaces, respectively; and
   circuitry, which (a) is attached to the housing, (b) is electrically coupled to the first and the second electrode surfaces, and (c) is configured to drive a current between the first and the second electrode surfaces.

For some applications, the at least a portion is less than the entire external surface of the drug capsule.

For some applications, the drug capsule includes a capsule selected from, the group consisting of: a hard-shelled capsule and a soft-shelled capsule.

For some applications, the at least a portion is the entire external surface of the drug capsule.

For some applications, the housing is a first housing, and the sensing apparatus further includes a second housing, which is sized and shaped to engage the first housing.

For some applications, the circuitry is attached inside the housing. For some applications, the circuitry is attached outside the housing.

For some applications, a shortest path between the first and the second electrode surfaces that does not pass through any elements of the apparatus is at least 4 mm. For some applications, the shortest path is no more than 20 mm.

For some applications, the circuitry is attached to the hemispherical portion of the housing.

For some applications, the first electrode surface is attached to the hemispherical portion.

For some applications, the second electrode surface is attached to the housing within 3 mm of a far end of the cylindrical portion from the hemispherical portion.

For some applications, the first electrode surface is attached to the hemispherical portion.

For some applications, the first electrode surface is disposed inside the housing, and the second electrode surface is disposed outside the housing.

For some applications, the housing includes a material having an electrical resistance of at least 100 ohms.

For some applications, the housing includes gelatin. For some applications, the housing is non-biodegradable.

For some applications, the housing is configured, when

submerged in a liquid having a pH of 3, to remain attached to the circuitry for at least one minute.

For some applications, the drug capsule is configured, when submerged in a liquid having a pH of 3, to dissolve to release the oral drug in a first amount of time, and the housing is configured, when submerged in the liquid having the pH of 3, to remain attached to the circuitry for at least a second amount of time greater than the first amount of time.

There is further provided, in accordance with an application of the present invention, apparatus for use with a disk-shaped drug tablet (a) having two major opposing surfaces connected by a cylindrical side wall and (b) including an oral drug, the apparatus including:

a housing, which is shaped so as to define a ring for tight fitting around an external surface of the cylindrical side wall of the drug tablet; and

a sensor, which includes:
first and second electrodes, which include first and second electrode surfaces, respectively; and
circuitry, which (a) is attached to the housing, (b) is electrically coupled to the first and the second electrode surfaces, and (c) is configured to drive a current between the first and the second electrode surfaces.

For some applications, the first and the second electrode surfaces are disposed on the ring at least 160 degrees from each other around the ring.

For some applications, the first and the second electrode surfaces are disposed on the ring 180 degrees from each other around the ring.

For some applications, the first and the second electrode surfaces are disposed on the ring at a closest distance of at least 4 mm from each other.

For some applications, the ring includes a hydrogel that is configured to undergo expansion upon contact with a liquid, and to increase a closest distance between the first and the second electrode surfaces upon the expansion.

For some applications:

the ring is configured, when submerged in a liquid having a pH of 3, to dissolve first at a first circumferential location around the ring,

the first electrode surface is disposed on the ring at a second circumferential location less than 45 degrees clockwise from the first circumferential location, and

the second electrode surface is disposed on the ring at a third circumferential location less than 45 degrees counterclockwise from the first circumferential location, and

the ring is configured to open and become straighter upon dissolving at the first circumferential location.

For some applications, an inner perimeter of the ring is between 3 and 15 mm, and a thickness of less than 8 mm.

For some applications, the ring includes a hydrogel that is configured to undergo expansion upon contact with a liquid, and to increase a closest distance between the first and the second electrode surfaces upon expansion.

For some applications, the apparatus further includes the disk-shaped drug tablet, and the ring is tightly fitted around the external surface of the cylindrical side wall of the drug tablet.

There is still further provided, in accordance with an application of the present invention, apparatus for use with an oral dosage form, the apparatus including a sensor, which is configured to assume compressed and expanded configurations, and which includes:

first and second electrodes, which include first and second electrode surfaces, respectively; and

circuitry, which is electrically coupled to the first and the second electrode surfaces,

wherein the sensor is:
(i) configured such that when the sensor is in the compressed configuration, the first and the second electrode surfaces are disposed at a closest compressed-configuration distance from each other, and
(ii) configured such that when the sensor is in the expanded configuration, the first and the second electrode surfaces are disposed at a closest expanded-configuration distance from each other, the closest expanded-configuration distance equal to at least 2 times the closest compressed-configuration distance, and
(iii) configured to drive a current between the first and the second electrode surfaces when the sensor is in the expanded configuration.

For some applications, the closest expanded-configuration distance equals at least 3 times the closest compressed-configuration distance.

For some applications, the sensor is constrained when in the compressed configuration, and unconstrained when in the expanded configuration.

For some applications, the first and the second electrode surfaces surface have shape memories, which are configured to transition the sensor from the compressed configuration to the expanded configuration.

For some applications, the sensor includes a hydrogel, which is configured to undergo expansion upon contact with a liquid, thereby transitioning the sensor from the compressed configuration to the expanded configuration, and increasing a closest distance between the first and the second electrode surfaces from, the closest compressed-configuration distance to the closest expanded-configuration distance.

For some applications:

the first electrode further includes a first elongate support structure, which is coupled to the circuitry at a first-structure coupling site along the first elongate support structure,

the first electrode surface is (A) electrically coupled to the circuitry via the first elongate support structure, and (B) disposed at a first electrode site along the first elongate support structure, wherein, when the sensor is in the expanded configuration, the first electrode site is disposed (x) within 2 mm of an end of the first elongate support structure, measured along the first elongate support structure, and (y) at least 3 mm from the first-structure coupling site, measured along the first elongate support structure,

the second electrode further includes a second elongate support structure, which is coupled to the circuitry at a second-structure coupling site along the second elongate support structure, and

the second electrode surface is (A) electrically coupled to the circuitry via the second elongate support structure, and (B) disposed at a second electrode site along the second elongate support structure, wherein, when the sensor is in the expanded configuration, the second electrode site is disposed (x) within 2 mm of an end of the second elongate support structure, measured along the second elongate support structure, and (y) at least 3 mm from the second-structure coupling site, measured along the second elongate support structure.

For some applications:

the sensor includes a hydrogel, which is configured to undergo expansion upon contact with a liquid, thereby transitioning the sensor from the compressed configuration to the expanded configuration, and

the first and the second elongate support structures are arranged such that the expansion of the hydrogel increases a closest distance between the first and the second electrode surfaces from the closest compressed-configuration distance to the closest expanded-configuration distance.

For some applications, the first and the second elongate support structures and the circuitry are embedded in the hydrogel. For some applications, an expanded volume of the hydrogel equals at least 1.5 times a compressed volume of the hydrogel. For some applications, the hydrogel is generally spherical when the sensor is in both the compressed configuration and the expanded configuration. For some applications, the apparatus further includes the oral dosage form, which includes a drug capsule, in which the sensor is disposed.

For some applications, the apparatus further includes the oral dosage form.

There is additionally provided, in accordance with an application of the present invention, apparatus including:

an oral dosage form; and

a sensing apparatus, which includes:
a housing, which is shaped so as to define a surface that is attached to at least a portion of an external surface of the oral dosage form by friction;
a sensor, which includes:
   first and second electrodes, which include first and second electrode surfaces, respectively; and
   circuitry, which (a) is attached to the housing, (b) is electrically coupled to the first and the second electrode surfaces, and (c) is configured to drive a current between the first and the second electrode surfaces.

There is yet additionally provided, in accordance with an application of the present invention, a method including:

receiving, by a human subject, (a) a drug capsule containing an oral drug and (b) a sensing apparatus, which includes (i) a housing, which is shaped so as to define exactly one hemispherical portion and exactly one cylindrical portion, which together define an internal surface tightly fitted to at least a portion of an external surface of the drug capsule, and (ii) a sensor, which includes (A) first and second electrodes, which include first and second electrode surfaces, respectively, and (B) circuitry, which (a) is attached to the housing, (b) is electrically coupled to the first, and the second electrode surfaces, and (c) is configured to drive a current between the first and the second electrode surfaces; and

swallowing, by the human subject, (a) the drug capsule and (b) the sensing apparatus while the internal surface is tightly fitted to the at least a portion of the external surface of the drug capsule.

For some applications, the at least a portion is less than the entire external surface of the drug capsule. For other applications, the at least a portion is the entire external surface of the drug capsule.

For some applications, the housing is a first housing, and the sensing apparatus further includes a second housing, which is sized and shaped to engage the first housing.

For some applications, receiving the drug capsule and the sensing apparatus includes receiving the drug capsule and the sensing apparatus while the circuitry is attached inside the housing. For some applications, receiving the drug capsule and the sensing apparatus includes receiving the drug capsule and the sensing apparatus while the circuitry is attached outside the housing.

For some applications, receiving the drug capsule and the sensing apparatus includes receiving the drug capsule and the sensing apparatus while a shortest path between the first and the second electrode surfaces that does not pass through any elements of the method is at least 4 mm.

For some applications, the shortest path is no more than 20 mm.

For some applications, receiving the drug capsule and the sensing apparatus includes receiving the drug capsule and the sensing apparatus while the circuitry is attached to the hemispherical portion of the housing.

For some applications, receiving the drug capsule and the sensing apparatus includes receiving the drug capsule and the sensing apparatus while the first electrode surface is attached to the hemispherical portion.

For some applications, receiving the drug capsule and the sensing apparatus includes receiving the drug capsule and the sensing apparatus while the second electrode surface is attached to the housing within 3 mm of a far end of the cylindrical portion from the hemispherical portion.

For some applications, receiving the drug capsule and the sensing apparatus includes receiving the drug capsule and the sensing apparatus while the first electrode surface is attached to the hemispherical portion.

For some applications, receiving the drug capsule and the sensing apparatus includes receiving the drug capsule and the sensing apparatus while the first electrode surface is disposed inside the housing, and the second electrode surface is disposed outside the housing.

For some applications, the housing includes a material having an electrical resistance of at least 100 ohms.

For some applications, the housing includes gelatin. For some applications, the housing is non-biodegradable.

For some applications, the housing is configured, when submerged in a liquid having a pH of 3, to remain attached to the circuitry for at least one minute.

For some applications, the drug capsule is configured, when submerged in a liquid having a pH of 3, to dissolve to release the oral drug in a first amount of time, and the housing is configured, when submerged in the liquid having the pH of 3, to remain attached to the circuitry for at least a second amount of time greater than the first amount of time.

There is also provided, in accordance with an application of the present invention, a method of assembly including:

providing (a) a drug capsule containing an oral drug and (b) a sensing apparatus, which includes (i) a housing, which is shaped so as to define exactly one hemispherical portion and exactly one cylindrical portion, which together define an internal surface for tight fitting to at least a portion of an external surface of the drug capsule, and (ii) a sensor, which includes (A) first and second electrodes, which include first and second electrode surfaces, respectively, and (B) circuitry, which (a) is attached to the housing, (b) is electrically coupled to the first and the second electrode surfaces, and (c) is configured to drive a current between the first and the second electrode surfaces;

holding the drug capsule; and

attaching the sensing apparatus to the drug capsule by tightly fitting the internal surface to the at least a portion of the external surface of the drug capsule.

For some applications, providing the drug capsule containing the oral drug includes filling the drug capsule with the oral drug.

There is further provided, in accordance with an application of the present invention, a method including:

receiving, by a human subject, (a) a disk-shaped drug tablet (i) having two major opposing surfaces connected by a cylindrical side wall and (ii) including an oral drug, and (b) a sensing apparatus, which includes (i) a housing, which is shaped so as to define a ring tightly fitted around an external surface of the cylindrical side wall of the drug tablet, and (ii) a sensor, which includes (A) first and second electrodes, which include first and second electrode surfaces, respectively, and (B) circuitry, which (a) is attached to the housing, (b) is electrically coupled to the first and the second electrode surfaces, and (c) is configured to drive a current between the first and the second electrode surfaces; and

swallowing, by the human subject, (a) the disk-shaped drug tablet and (b) the sensing apparatus while the ring is tightly fitted around the external surface of the cylindrical side wall of the drug tablet.

For some applications, receiving the disk-shaped drug tablet and the sensing apparatus includes receiving the disk-shaped drug tablet and the sensing apparatus while the first and the second electrode surfaces are disposed on the ring at least 160 degrees from, each other around the ring.

For some applications, receiving the disk-shaped drug tablet and the sensing apparatus includes receiving the disk-shaped drug tablet and the sensing apparatus while the first and the second electrode surfaces are disposed on the ring 180 degrees from each other around the ring.

For some applications, receiving the disk-shaped drug tablet and the sensing apparatus includes receiving the disk-shaped drug tablet and the sensing apparatus while the first and the second electrode surfaces are disposed on the ring at a closest distance of at least 4 mm from, each other.

For some applications, receiving the disk-shaped drug tablet and the sensing apparatus includes receiving the disk-shaped, drug tablet and the sensing apparatus while the ring includes a hydrogel that is configured to undergo expansion upon contact with a liquid, and to increase a closest distance between the first and the second electrode surfaces upon the expansion.

For some applications:

the ring is configured, when submerged in a liquid having a pH of 3, to dissolve first at a first circumferential location around the ring,

receiving the disk-shaped drug tablet and the sensing apparatus includes receiving the disk-shaped, drug tablet and the sensing apparatus while (a) the first electrode surface is disposed on the ring at a second circumferential location less than 45 degrees clockwise from the first circumferential location, and (b) the second electrode surface is disposed on the ring at a third circumferential location less than 45 degrees counterclockwise from, the first circumferential location, and

the ring is configured to open and become straighter upon dissolving at the first circumferential location.

For some applications, receiving the disk-shaped drug tablet and the sensing apparatus includes receiving the disk-shaped drug tablet and the sensing apparatus while an inner perimeter of the ring is between 3 and 15 mm, and a thickness of less than 8 mm.

For some applications, receiving the disk-shaped drug tablet and the sensing apparatus includes receiving the disk-shaped drug tablet and the sensing apparatus while the ring includes a hydrogel that is configured to undergo expansion upon contact with a liquid, and to increase a closest distance between the first and the second electrode surfaces upon expansion.

There is still further provided, in accordance with an application of the present invention, a method of assembly including:

providing (a) a disk-shaped drug tablet (i) having two major opposing surfaces connected by a cylindrical side wall and (ii) including an oral drug, and (b) a sensing apparatus, which includes (i) a housing, which is shaped so as to define a ring for tight fitting around an external surface of the cylindrical side wall of the drug tablet, and (ii) a sensor, which includes (A) first and second electrodes, which include first and second electrode surfaces, respectively, and (B) circuitry, which (a) is attached to the housing, (b) is electrically coupled to the first and the second electrode surfaces, and (c) is configured to drive a current between the first and the second electrode surfaces;

holding the drug tablet; and

attaching the sensing apparatus to the disk-shaped drug tablet by tightly fitting the ring around the external surface of the cylindrical side wall of the drug tablet.

There is additionally provided, in accordance with an application of the present invention, a method including:

receiving, by a human subject, (a) an oral dosage form and (b) a sensor, which is in a compressed configuration, and which includes (i) first and second electrodes, which include first and second electrode surfaces, respectively, and (ii) circuitry, which is electrically coupled to the first and the second electrode surfaces, wherein when the sensor is in the compressed configuration, the first and the second electrode surfaces are disposed at a closest compressed-configuration distance from each other; and

swallowing, by the human subject, (a) the oral dosage form and (b) the sensor while the sensor is coupled to the oral dosage form, such that the sensor, upon contact with gastric acid in a stomach of the human subject, transitions from the compressed configuration to an expanded configuration, in which (i) the first and the second electrode surfaces are disposed at a closest expanded-configuration distance from each other, the closest expanded-configuration distance equal to at least 2 times the closest compressed-configuration distance, and (ii) the sensor is configured to drive a current between the first and the second electrode surfaces.

For some applications, the closest expanded-configuration distance equals at least 3 times the closest compressed-configuration distance.

For some applications:

receiving the oral dosage form and the sensor includes receiving the oral dosage form and the sensor while the sensor is constrained in the compressed configuration, and

swallowing the oral dosage form and the sensor includes swallowing the receiving the oral dosage form and the sensor such that that the sensor becomes unconstrained in the expanded configuration.

For some applications, the first and the second electrode surfaces surface have shape memories, which are configured to transition the sensor from the compressed configuration to the expanded configuration.

For some applications, the sensor includes a hydrogel, which is configured to undergo expansion upon contact with the gastric acid, thereby transitioning the sensor from the compressed configuration to the expanded configuration, and increasing a closest distance between the first and the second electrode surfaces from the closest compressed-configuration distance to the closest expanded-configuration distance.

For some applications:

the first electrode further includes a first elongate support structure, which is coupled to the circuitry at a first-structure coupling site along the first elongate support structure,

the first electrode surface is (A) electrically coupled to the circuitry via the first elongate support structure, and (B) disposed at a first electrode site along the first elongate support structure, wherein, when the sensor is in the expanded configuration, the first electrode site is disposed (x) within 2 mm of an end of the first elongate support structure, measured along the first elongate support structure, and (y) at least 3 mm from the first-structure coupling site, measured along the first, elongate support, structure,

the second electrode further includes a second elongate support structure, which is coupled to the circuitry at a second-structure coupling site along the second elongate support structure, and

the second electrode surface is (A) electrically coupled to the circuitry via the second elongate support structure, and (B) disposed at a second electrode site along the second elongate support structure, wherein, when the sensor is in the expanded configuration, the second electrode site is disposed (x) within 2 mm of an end of the second elongate support structure, measured along the second elongate support structure, and (y) at least 3 mm from the second-structure coupling site, measured along the second elongate support structure.

For some applications:

the sensor includes a hydrogel, which is configured to undergo expansion upon contact with the gastric acid, thereby transitioning the sensor from the compressed configuration to the expanded configuration, and

the first and the second elongate support structures are arranged such that the expansion of the hydrogel increases a closest distance between the first and the second electrode surfaces from the closest compressed-configuration distance to the closest expanded-configuration distance.

For some applications, the first and the second elongate support structures and the circuitry are embedded in the hydrogel.

For some applications, an expanded volume of the hydrogel equals at least 1.5 times a compressed volume of the hydrogel.

For some applications, the hydrogel is generally spherical when the sensor is in both the compressed configuration and the expanded configuration.

There is yet additionally provided, in accordance with an application of the present invention, a method of assembly including:

providing (a) an oral dosage form and (b) a sensor, which is in a compressed configuration, and which includes (i) first and second electrodes, which include first and second electrode surfaces, respectively, and (ii) circuitry, which is electrically coupled to the first and the second electrode surfaces, wherein when the sensor is in the compressed configuration, the first and the second electrode surfaces are disposed at a closest compressed-configuration distance from each other, wherein the sensor is configured, upon contact with gastric acid in a stomach of a human subject, to transition from the compressed configuration to an expanded configuration, in which (i) the first and the second electrode surfaces are disposed at a closest expanded-configuration distance from each other, the closest expanded-configuration distance equal to at least 2 times the closest compressed-configuration distance, and (ii) the sensor is configured to drive a current between the first and the second electrode surfaces;

holding the oral dosage form; and

coupling the sensor to the oral dosage form.

There is also provided, in accordance with an application of the present invention, a method including:

receiving, by a human subject, (a) an oral dosage form, and (b) a sensing apparatus, which includes (i) a housing, which is shaped so as to define a surface that is attached to an external surface of the oral dosage form by friction, (ii) and a sensor, which includes (A) first and second electrodes, which include first and second electrode surfaces, respectively, and (B) circuitry, which (a) is attached to the housing, (b) is electrically coupled to the first and the second electrode surfaces, and (c) is configured to drive a current between the first and the second electrode surfaces; and

swallowing, by the human subject, (a) the oral dosage form and (b) the sensing apparatus while the surface is attached to the at least a portion of the external surface of the oral dosage form.

There is further provided, in accordance with an application of the present invention, a method of assembly including:

providing (a) an oral dosage form, and (b) a sensing apparatus, which includes (i) a housing, which is shaped so as to define a surface that is attachable to at least a portion of an external surface of the oral dosage form by friction, and (ii) a sensor, which includes (A) first and second electrodes, which include first and second electrode surfaces, respectively, and (B) circuitry, which (a) is attached to the housing, (b) is electrically coupled to the first and the second electrode surfaces, and (c) is configured to drive a current between the first and the second electrode surfaces;

holding the oral dosage form; and

attaching the sensing apparatus to the oral dosage form by friction.

The present invention will be more fully understood from the following detailed description of embodiments thereof, taken together with the drawings, in which:

### BRIEF DESCRIPTION OF THE DRAWINGS

FIGS. 1A-B are schematic illustrations of sensing apparatus and a drug capsule containing an oral drug, in accordance with an application of the present invention;

FIG. 2 is a bottom-view of the sensing apparatus of FIGS. 1A-B before attachment to the drug capsule of FIGS. 1A-B, in accordance with an application of the present invention;

FIGS. 3A-B are schematic illustrations of an alternative configuration of the sensing apparatus of FIG. 1A-B, in accordance with an application of the present invention;

FIG. 4A is a schematic illustration of another configuration of the sensing apparatus of FIGS. 1A-B and 2, in accordance with an application of the present invention;

FIG. 4B is a schematic illustration of yet another configuration of the sensing apparatus of FIGS. 1A-B and 2, in accordance with an application of the present invention;

FIG. 5 is a schematic illustration of the sensing apparatus of FIGS. 1A-B and 2 after having been swallowed by a human subject, in accordance with an application of the present invention;

FIGS. 6A-B are schematic illustrations of sensing apparatus for use with a disk-shaped drug tablet, in accordance with an application of the present invention;

FIG. 7 is a schematic illustration of another configuration of the sensing apparatus of FIGS. 6A-B, in accordance with an application of the present invention;

FIGS. 8A-B are schematic illustrations of yet another configuration of the sensing apparatus of FIGS. 6A-B, in accordance with an application of the present invention;

FIGS. 9A-B are schematic illustrations of still another configuration of the sensing apparatus of FIGS. 6A-B, in accordance with an application of the present invention; and

FIGS. 10A-B are schematic illustrations of another sensing apparatus for use with an oral dosage form, in accordance with an application of the present invention.

### DETAILED DESCRIPTION OF APPLICATIONS

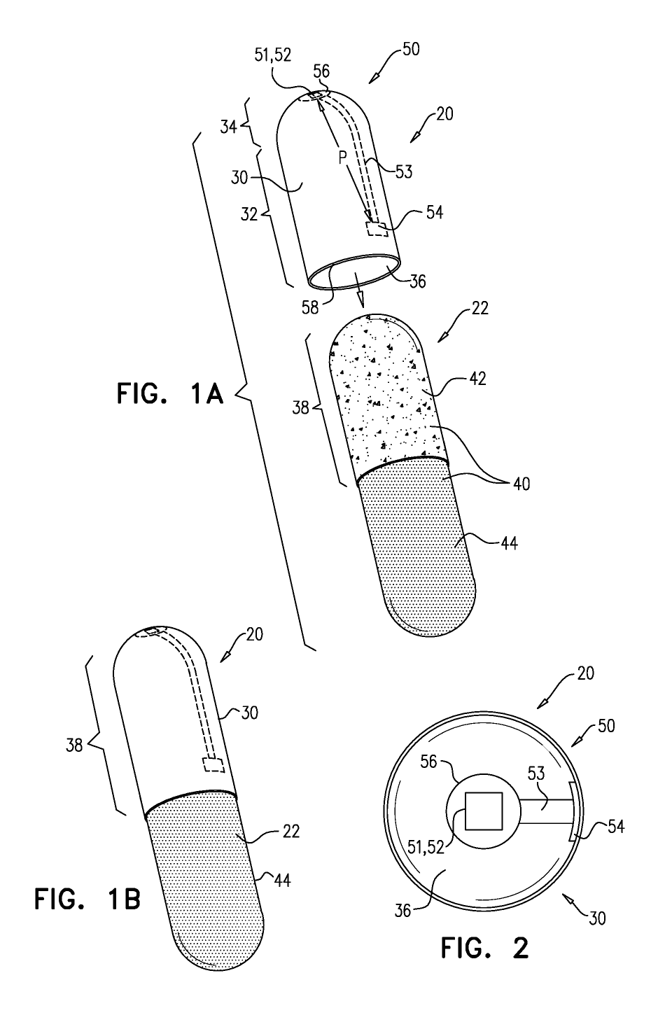

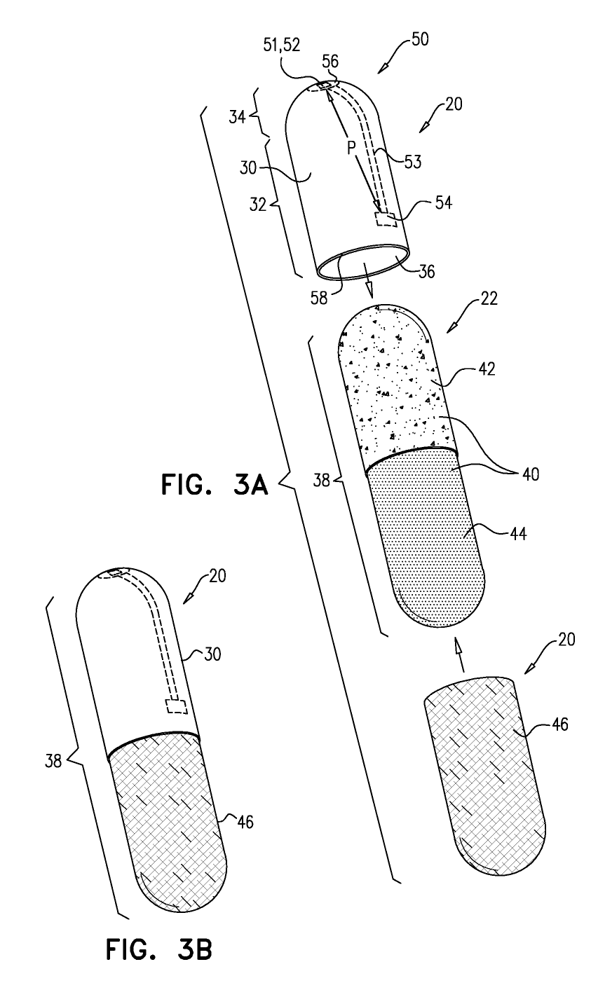

FIGS. 1A-B, 2, and 3A-B are schematic illustrations of sensing apparatus 20 and a drug capsule 22 containing an oral drug, in accordance with respective applications of the present invention. FIGS. 1A and 1B show sensing apparatus 20 before and after attachment to drug capsule 22, respectively. FIG. 2 is a bottom-view of sensing apparatus 20 before attachment to drug capsule 22. FIGS. 3A and 3B show an alternative configuration of sensing apparatus 20 before and after attachment to drug capsule 22, respectively. Alternatively, drug capsule 22 contains a placebo. For some applications, sensing apparatus 20 is provided (e.g., sold or distributed) with drug capsule 22, while for other applications, sensing apparatus 20 is provided (e.g., sold or distributed) without drug capsule 22, i.e., an apparatus may be provided that comprises sensing apparatus 20 and not drug capsule 22. Although drug capsule 22 is illustrated as a hard-shelled capsule comprising two pieces fitted together (a cap 42 and a body 44 (i.e., the lower-diameter half)), drug capsule 22 may alternatively comprise a soft-shelled capsule (also known as a gel capsule), which typically comprises a single piece that contains the oral drug therein. Alternatively, for some applications, sensing apparatus 20 is used with an oblong drug tablet, which may be shaped like a capsule.

Sensing apparatus 20 is configured emit a detectable signal upon contact with a target physiological liquid inside a body of a human subject, such as gastric acid, after drug capsule 22 has been swallowed with sensing apparatus 20 attached thereto. The signal is detectable by a separate sensing unit, which is typically configured to be disposed external the subject's body, such as described hereinbelow with reference to FIG. 5.

Sensing apparatus 20 comprises a housing 30, which is shaped so as to define exactly one hemispherical portion 34 and exactly one cylindrical portion 32, which together define an internal surface 36 for tight fitting to at least a portion 38 of an external surface 40 of drug capsule 22. Optionally, internal surface 36 of housing 30 is attached to the at least a portion 38 by friction.

For some applications, such as shown in FIGS. 1A-B and 5, the at least a portion 38 of external surface 40 of drug capsule 22 is less than the entire external surface 40 of drug capsule 22. For some applications, the at least a portion 38 of external surface 40 of drug capsule 22 comprises a cap 42 of drug capsule 22, such as shown (and may optionally include a portion of body 44 of drug capsule 22). For other applications, the at least a portion 38 of external surface 40 of drug capsule 22 comprises body 44 of drug capsule 22 (and may optionally include a portion of cap 42 of drug capsule 22) (configuration not shown). For still other applications, cap housing 30 itself is shaped as (a) the cap of drug capsule 22, in which case the at least a portion 38 of external surface 40 of drug capsule 22 is the portion of body 44 of drug capsule 22 that overlaps housing 30, or (b) the body of drug capsule 22, in which case the at least a portion 38 of external surface 40 of drag capsule 22 is the portion of cap 42 of drug capsule 22 that overlaps housing 30.

For other applications, such as shown in FIGS. 3A-B, the at least a portion 38 of external surface 40 of drug capsule 22 is the entire external surface 40 of drug capsule 22. For some of these applications, housing 30 is a first housing 30 that comprises one piece of a two-piece capsule (either a cap (as shown) or a body (i.e., the lower-diameter half) (configuration not shown)), and sensing apparatus 20 further comprises a second housing 46, which is sized and shaped to engage first housing 30. For some applications, second housing 46 is shaped so as to define exactly one hemispherical portion and exactly one cylindrical portion, as shown. For some applications, first and second housings 30 and 46 together surround and contain drug capsule 22, as shown in FIG. 3B.

Sensing apparatus 20 further comprises a sensor 50, which comprises:

first and second electrodes 51 and 53, which comprise first and second electrode surfaces 52 and 54, respectively; and

circuitry 56, which (a) is attached to housing 30, (b) is electrically coupled to first and second electrode surfaces 52 and 54, and (c) is configured to drive a current between first and second electrode surfaces 52 and 54.

First and second electrode surfaces 52 and 54 are the interface portions of first and second electrodes 51 and 53 through which current can flow from the electrodes to the environment surrounding the electrodes.

The driving of the current between first and second electrode surfaces 52 and 54 emits a detectable signal. For some applications, circuitry 56 is configured to generate the detectable signal as a series of pulses. For some applications, circuitry 56 is configured to intermittently short the electrodes. (Typically, first and second electrodes 51 and 53 do not function as an antenna.)

As used in the specification, including in the claims “electrically coupled” means electrically coupled over one or more conductive elements of the sensing apparatus, such as wires, and does not include wirelessly coupled. For some applications, sensor 50 comprises a plurality of sets of first and second electrodes 51 and 53, and, optionally, separate circuitry 56 for each set.

For some applications, sensor 50 (and sensing apparatus 20) does not comprise a complete battery when initially coupled to drug capsule 22, prior to being swallowed. Instead, sensor 50 derives energy from an acid (such as gastric acid) when sensor 50 comes in contact with the acid, e.g., is submerged in the acid, such as when sensing apparatus 20 is disposed in the stomach upon being swallowed. Thus, contact with the acid (such as gastric acid) activates circuitry 56.

For some applications, first and second electrodes 51 and 53 are configured to function as a cathode and an anode, respectively, or vice versa, and the gastric acid functions as an electrolyte, such that the cathode, anode, and electrolyte together operate as a complete battery that generates a voltage between the electrodes, when first and second electrode surfaces 52 and 54 come in contact with the gastric acid in the stomach. First and second electrodes 51 and 53 comprise two dissimilar electrochemical materials. For example, the anode may comprise Mg+ or Ca++, and the cathode may comprise AgCl, or other suitable combinations of biocompatible, non-toxic materials as is known in the battery art.

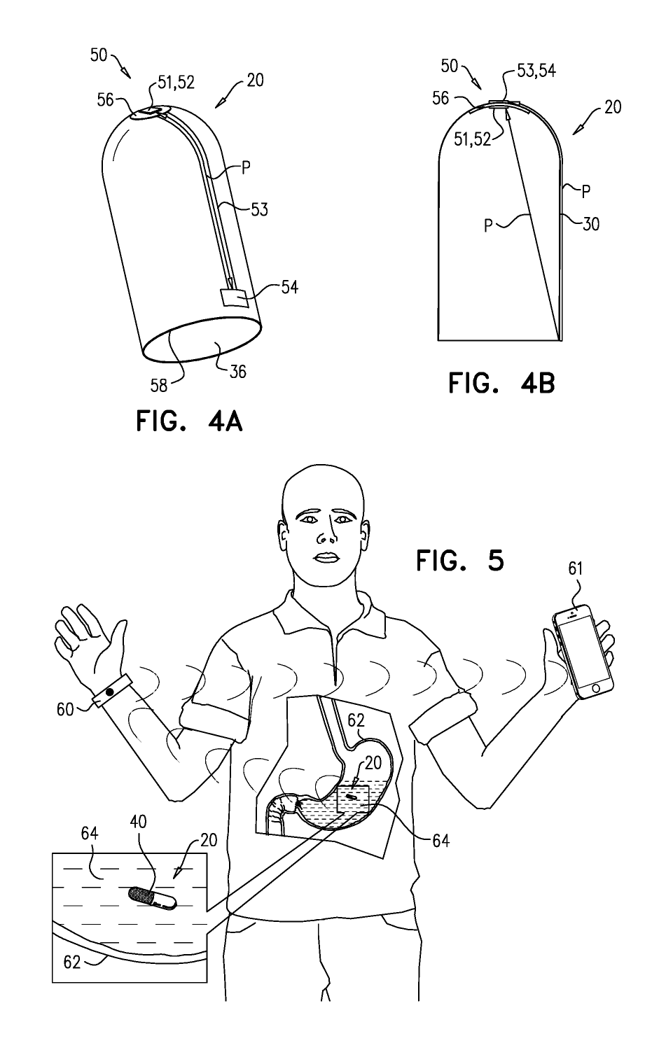

For some applications, first and second electrode surfaces 52 and 54 (or the other electrode surfaces described hereinbelow in other configurations) are coated with a biocompatible coating, which is configured to dissolve when submerged in a liquid having a pH of 3 (such as gastric acid). Dissolving of the coating exposes first and second electrode surfaces 52 and 54 to the acid, which functions as an electrolyte and generates a voltage, as described above. In addition, in some configurations, such as those shown in FIGS. 4A and 4B, the coating prevents the visible exposure of metal on an external surface of housing 30, which may lead to corrosion of the metal and/or be unappealing to patients.

For some applications, housing 30 comprises gelatin. For some applications, housing 30 is non-biodegradable. For some applications, housing 30 is configured, when submerged in a liquid having a pH of 3 (such as gastric acid), to remain attached to circuitry 56 for at least one minute, and/or sufficient time for the electrodes to begin generating a voltage and sensing apparatus 20 to generate the signal, as discussed above. For some applications, drug capsule 22 is configured, when submerged in a liquid having a pH of 3, to dissolve to release the oral drug in a first amount of time, and wherein housing 30 is configured, when submerged in the liquid having the pH of 3, to remain attached to circuitry 56 (and, typically, electrically-insulating) for at least a second amount of time greater than the first amount of time, such as at least 50% greater and/or 30 seconds, e.g., one minute, longer. This greater amount of time allows for the establishment and maintenance of the shortest path between the first and the second electrode surfaces, as described hereinbelow.

Reference is still made to FIGS. 1A-B, 2, and 3A-B, and is additionally made to FIG. 4A, which is a schematic illustration of another configuration of sensing apparatus 20, in accordance with an application of the present invention. For some applications, such as shown in 1A-B, 2, and 3A-B, circuitry 56 is attached inside housing 30, while for other applications, such as shown in FIG. 4A, circuitry 56 is attached outside housing 30. Alternatively or additionally, for some applications, such as shown in FIGS. 1A-B, 2, 3A-B, and 4A, circuitry 56 is attached to hemispherical portion 34 of housing 30.

For some applications, such as shown in FIGS. 1A-B, 2, 3A-B, and 4A, second electrode surface 54 is attached to housing 30 within 3 mm of (e.g., with 2 mm of, or within 1 mm of, such as at) a far end 58 of cylindrical portion 32 from hemispherical portion 34. Alternatively or additionally, for some applications, such as shown in FIGS. 1A-B, 2, 3A-B, and 4A-B, first electrode surface 52 is attached to hemispherical portion 34.

Reference is made to FIG. 4B, which is a schematic illustration of yet another configuration of sensing apparatus 20, in accordance with an application of the present invention. For some applications, first electrode surface 52 is disposed inside housing 30, and second electrode surface 54 is disposed outside housing 30. Typically, both first and second electrode surfaces 52 and 54 are attached to hemispherical portion 34 of housing 30, such as shown. Alternatively, one or more of first and second electrode surfaces 52 and 54 are attached to cylindrical portion 32, For some applications, housing 30 comprises a material having an electrical resistance of at least 100 ohms.

For some applications, a shortest path P between first and second electrode surfaces 52 and 54 that does not pass through any elements of sensing apparatus 20 is at least 4 mm (such as at least 6 mm), no more than 20 mm (such as no more than 10 mm), and/or between 4 and 20 mm (such as between 6 and 10 mm). Disposition of first and second electrode surfaces 52 and 54 at these effective distances from, each other increases the distance from which the signal generated by the circuitry 56 can be readily detected, such as by sensing unit 60, described hereinbelow with reference to FIG. 5. Typically, housing 30 comprises an electrically-insulating material, which lengths shortest path P, thereby increasing the effective distance between the electrode surfaces. (For clarity of illustration, a portion of shortest path P is shown in FIG. 4B as not quite touching the external surface of housing 30, although in reality this portion of shortest path P is measured on the external surface of housing 30.)

Reference is now made to FIG. 5, which is a schematic illustration of sensing apparatus 20 after having been swallowed by a human subject while sensing apparatus 20 while internal surface 36 is tightly fitted to the at least a portion 38 of external surface 40 of drug capsule 22, in accordance with an application of the present invention. In this application, a sensing unit 60 is provided, which comprises circuitry configured to sense the signal emitted by sensing apparatus 20. Typically, sensing unit 60 is configured to be disposed external to the subject's body. For example, sensing unit 60 may be integrated into an article of clothing, such as a wristwatch, or provided on an adhesive patch, which may be placed on the subject's skin, e.g., on the front or back of the subject's torso, or on the subject's wrist or arm. For some applications, sensing unit 60 comprises electrodes, such as surface electrodes, e.g., EMG electrodes, as are known in the EMG art. For some applications, sensing unit 60 is configured to be coupled, either wirelessly or over wires, with a data processing unit, such as a smartphone or a wireless or wired network.

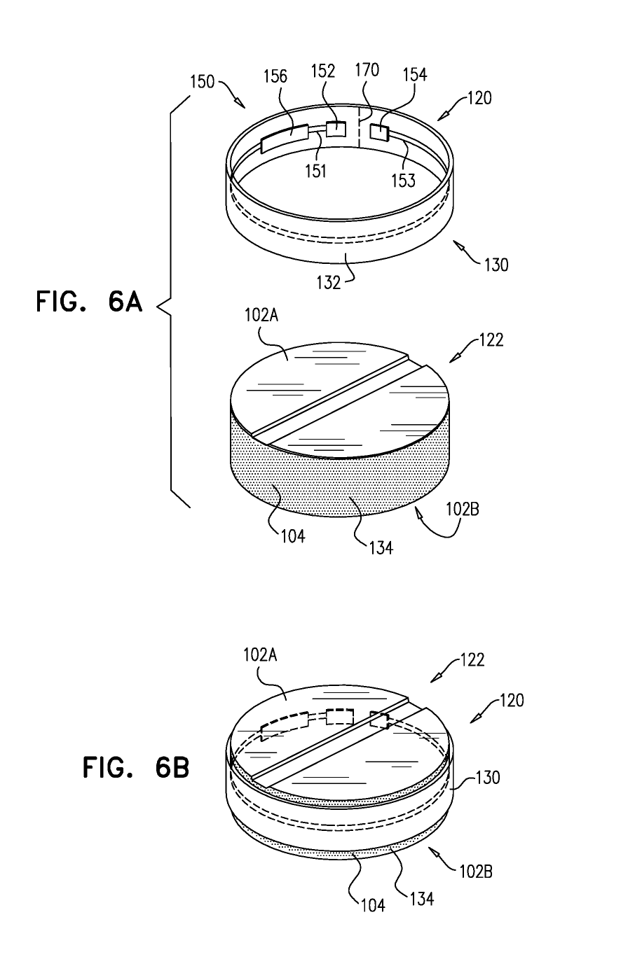

Reference is now made to FIGS. 6A-B, which are schematic illustrations of sensing apparatus 120 for use with a disk-shaped drug tablet 122, in accordance with an application of the present invention. FIGS. 6A and 6B show sensing apparatus 120 before and after attachment to drug tablet 122, respectively. Drug tablet 122 has two major opposing surfaces 102A and 102B connected by a cylindrical side wall 104, and comprises an oral drug. Alternatively, disk-shaped drug tablet 122 comprises a placebo.

Sensing apparatus 120 comprises a housing 130, which is shaped so as to define a ring 132 for tight fitting around an external surface 134 of cylindrical side wall 104 of drug tablet 122, The shape of ring 132 will depend on the shape of external surface 134; for example, ring 132 may be circular, as shown, or may have another shape, such as a regular polygon, e.g., a hexagon. For some applications, an inner perimeter of ring 132 is at least 3 mm, no more than 12 mm, and/or between 3 mm to 15 mm, such as at least 5 mm, no more than 10 mm, and/or between 5 and 10 mm, and a thickness of at least 1 mm, no more than 8 mm, and/or between 1 and 8 mm, such as at least 2 mm, no more than 5 mm, and/or between 2 and 5 mm. Optionally, ring 132 is attached to external surface 134 by friction.

Sensing apparatus 120 further comprises a sensor 150, which comprises:

first and second electrodes 151 and 153, which comprise first and second electrode surfaces 152 and 154, respectively; and

circuitry 156, which (a) is attached to housing 130, (b) is electrically coupled to first and second electrode surfaces 152 and 154, and (c) is configured to drive a current between first and second electrode surfaces 152 and 154, as described hereinabove regarding sensing apparatus 20 with reference to FIGS. 1A-B, 2, and 3A-B.

The driving of the current between first and second electrode surfaces 152 and 154 emits a detectable signal, such as described hereinabove with reference to FIGS. 1A-B, 2, and 3A-B. Circuitry 156 and/or electrodes 151 and 153 may be configured as described hereinabove with reference to FIGS. 1A-B, 2, and 3A-B regarding circuitry 56 and/or electrodes 51 and 53, mutatis mutandis. For some applications, sensor 150 comprises a plurality of sets of first and second electrodes 151 and 153, and, optionally, separate circuitry 156 for each set.

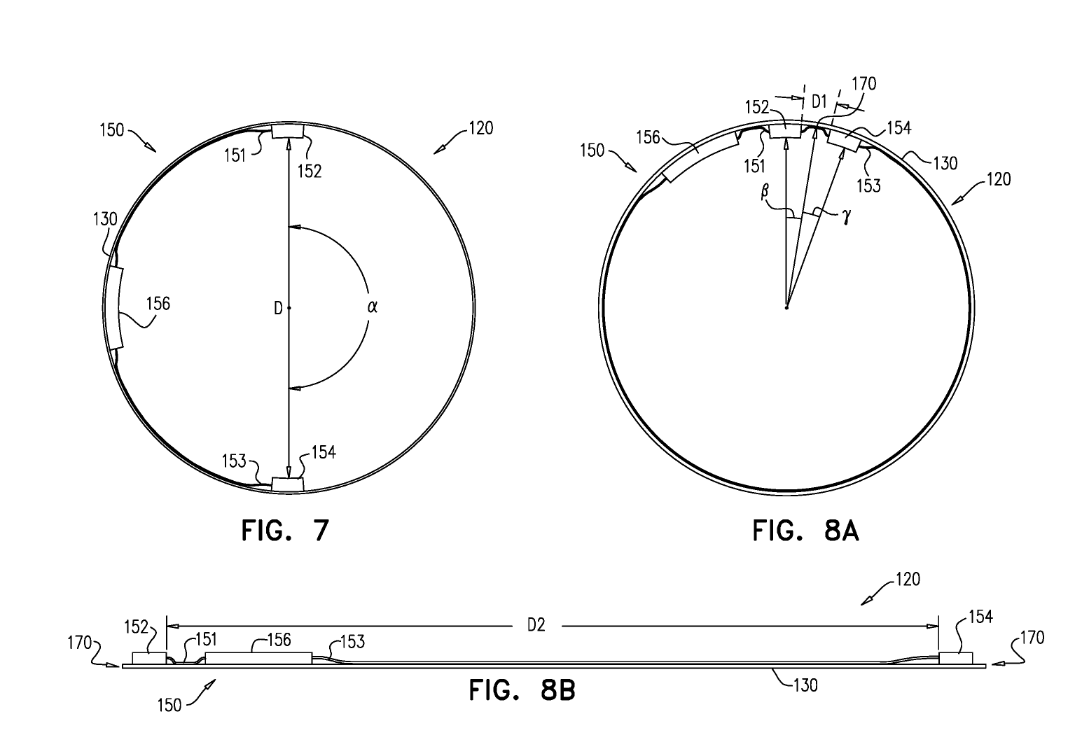

Reference is made to FIG. 7, which is a schematic illustration of another configuration of sensing apparatus 120, in accordance with an application of the present invention. In this configuration, first and second electrode surfaces 152 and 154 are disposed on ring 132 at angle α (alpha) of at least 160 degrees from each other around ring 132, such as 180 degrees (as shown). For some applications, first and second electrode surfaces 152 and 154 are disposed on ring 132 at a closest distance D of at least 4 mm (e.g., at least 6 mm, such as at least 8 mm) from each other (i.e., measured across a portion of the space surrounded by ring 132). As described hereinabove with reference to FIGS. 1A-5, disposition of first and second electrode surfaces 152 and 154 at a greater distance from each other increases the distance from which the signal generated by the circuitry can be readily detected, such as by sensing unit 60, described hereinabove with reference to FIG. 5.

Reference is now made to FIGS. 8A-B, which are schematic illustrations of yet another configuration of sensing apparatus 120, in accordance with an application of the present invention. FIG. 8A shows ring 132 in its initial configuration, in which it is attached to drug tablet 122 (for clarity of illustration, drug tablet 122 is not shown). FIG. 8B shows ring 132 after it has opened in the stomach, as described below.

In this configuration, ring 132 is configured, when submerged in a liquid having a pH of 3 (such as gastric acid), to dissolve first at a first circumferential location 170 around ring 132 (labeled in FIGS. 8A-B, as well as in FIG. 6A). First electrode surface 152 is disposed on ring 132 at a second circumferential location less than 45 degrees (e.g., less than 30 degrees, such as less than 15 degrees) counterclockwise from first circumferential location 170, and second electrode surface 154 is disposed on ring 132 at a third circumferential location less than 45 degrees (e.g., less than 30 degrees, such as less than 15 degrees) clockwise from first circumferential location 170. For example, first circumferential location 170, the second circumferential. location, and the third, circumferential location may be at 12 o'clock, 11:55, and 12:05, respectively. As shown in FIG. 8B, ring 132 is configured to open and become straighter upon dissolving at first circumferential location 170, which brings first and second electrode surface 152 and 154 farther from, each other than when in the initial configuration shown in FIG. 8A. In other words, first and second electrode surfaces 152 and 154 are disposed, at a closest initial-configuration distance D1 from each other, as shown in FIG. 8A, and a closest expanded-configuration distance D2 that is greater than closest initial-configuration distance D1, as shown in FIG. 8B. For some applications, closest expanded-configuration distance D2 equals at least 5 (e.g., at least 10, or at least 20) times closest initial-configuration distance D1, and/or at least 12 mm and/or no more than 31 mm.

For some applications, ring 132 is configured to become straighter upon dissolving at first circumferential location 170, because housing 130 and/or first and second electrodes 151 and 153 comprise a material having a shape memory (e.g., Nitinol).

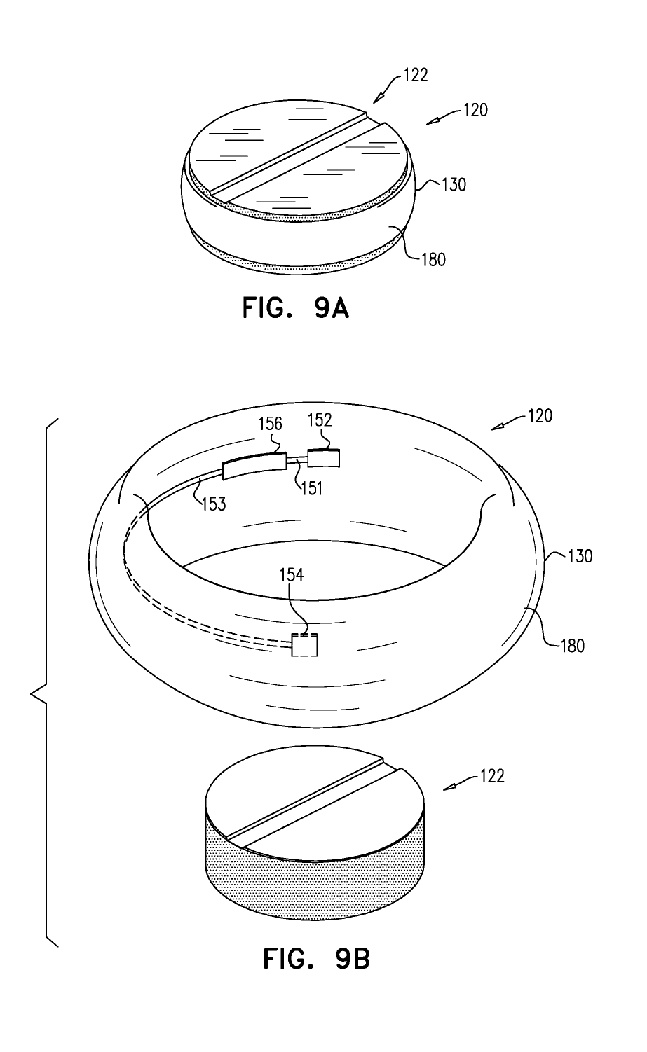

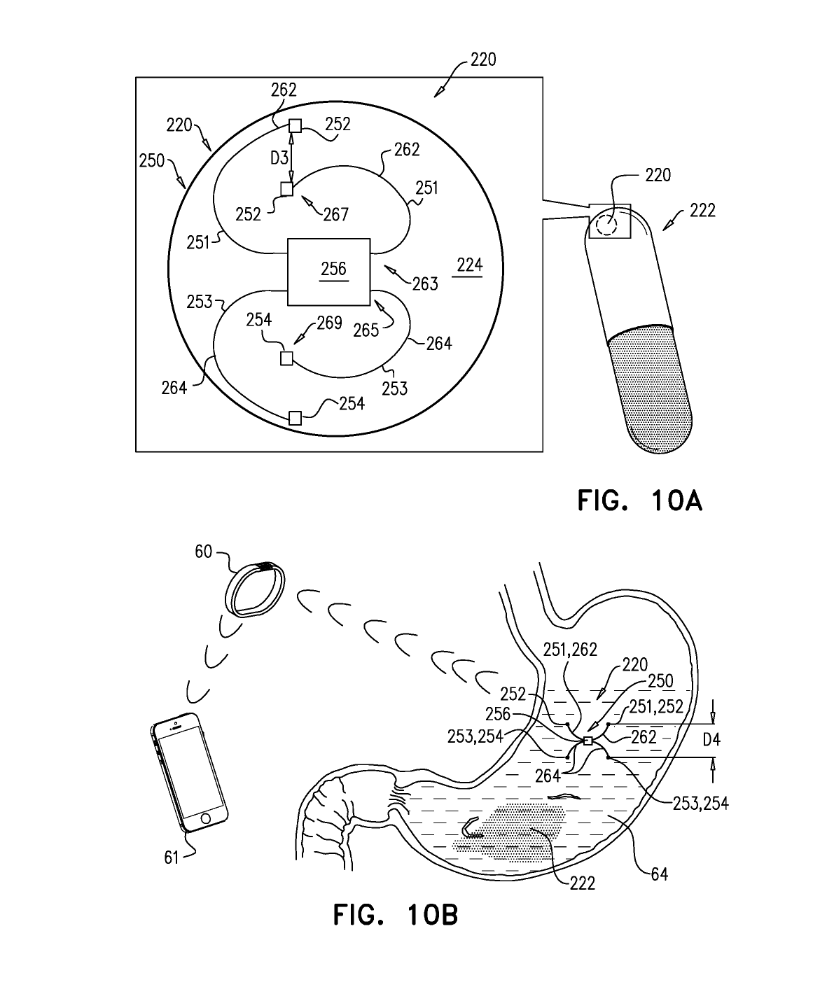

Reference is now made to FIGS. 9A-B, which are schematic illustrations of still another configuration of sensing apparatus 120, in accordance with an application of the present invention. FIGS. 8A and 8B show ring 132 respectively in (a) an initial unexpanded configuration, in which it is attached to drug tablet 122, and (b) an expanded configuration, in which it has become detached from drug tablet 122 (although drug tablet 122 is shown intact in the latter state, it may in practice have partially or completely dissolved by the time that ring 132 reaches the latter state).

In this configuration, ring 132 comprises a hydrogel 180 that is configured to undergo expansion upon contact with a liquid (e.g., having a pH of 3 (such as gastric acid)), and to increase a closest distance between first and second electrode surfaces 152 and 154 upon expansion. In other words, first and second electrode surfaces 152 and 154 are disposed at a closest initial-configuration distance from each other, as shown in FIG. 9A, and a closest expanded-configuration distance that is greater than the closest initial-configuration distance, as shown in FIG. 9B. For some applications, the closest expanded-configuration distance equals at least 1.5 times (e.g., 2 times) the closest initial-configuration distance. This configuration may implement any of the features described hereinabove with reference to FIG. 7.

Reference is now made to FIGS. 10A-B, which are schematic illustrations of another sensing apparatus 220 for use with an oral dosage form 222, in accordance with an application of the present invention. For example, oral dosage form 222 may comprise a drug capsule or a drug tablet, which, comprises a drug or a placebo. Sensing apparatus 220 comprises a sensor 250, which is configured to assume compressed and expanded configurations, as shown in FIGS. 10A and 10B, respectively. For some applications, sensor 250 is configured to transition from the compressed configuration to the expanded configuration upon contact with a liquid (e.g., having a pH of 3 (such as gastric acid). Sensing apparatus 220 may implement any of the techniques described hereinabove for sensing apparatus 20 and/or sensing apparatus 120, mutatis mutandis.

Sensor 250 is initially coupled to oral dosage form 222 when in the compressed configuration. For example, for applications in which oral dosage form 222 comprises a drug capsule (e.g., a hard-shelled or soft-shelled capsule), sensor 250 may, for example, be disposed within the drug capsule; for applications in which oral dosage form 222 comprises a drug tablet, sensor 250 may, for example, be attached to an external surface of the drug tablet.

Sensor 250 comprises (a) first and second electrodes 251 and 253, which comprise first and second electrode surfaces 252 and 254, respectively, and (b) circuitry 256, which is electrically coupled to first and second electrode surfaces 252 and 254. Sensor 250 is:

configured such that when sensor 250 is in the compressed configuration, such as shown in FIG. 10A, first and second electrode surfaces 252 and 254 are disposed at a closest compressed-configuration distance D3 from each other, and

configured such that when sensor 250 is in the expanded configuration, first and second electrode surfaces 252 and 254 are disposed at a closest expanded-configuration distance D4 from each other, the closest expanded-configuration distance D4 equal to at least 2 times (e.g., at least 3 times, or at least 4 times) the closest compressed-configuration distance D3, no more than 8 times (e.g., no more than 5 times) the closest compressed-configuration distance D3, and/or between 2 and 8 times (e.g., between 3 and 5 times) the closest compressed-configuration distance D3, and

configured to drive a current between first and second electrode surfaces 252 and 254, as described hereinabove regarding sensing apparatus 20 with reference to FIGS. 1A-B, 2, and 3A-B, when sensor 250 is in the expanded configuration, such as shown in FIG. 10B.

The driving of the current between first and second electrode surfaces 252 and 254 emits a detectable signal, such as described hereinabove with reference to FIGS. 1A-B, 2, and 3A-B. Circuitry 256 and/or electrodes 251 and 253 may be configured as described hereinabove with reference to FIGS. 1A-B, 2, and 3A-B regarding circuitry 56 and/or electrodes 51 and 53, mutatis mutandis. For some applications, sensor 250 comprises a plurality of sets of first and second electrodes 251 and 253, as shown, and, optionally, separate circuitry 56 for each set. For example, all of first electrodes 251 may comprise anodes, and all of second electrodes 253 may comprise cathodes, or vice versa.

For some applications, sensor 250 is constrained when in the compressed configuration, and unconstrained when in the expanded configuration.

For some applications, first and second electrode surfaces 252 and 254 have shape memories, which are configured to transition sensor 250 from the compressed configuration to the expanded configuration.

For some applications, sensor 250 comprises a hydrogel 244, which is configured to undergo expansion upon contact with a liquid (e.g., having a pH of 3 (such as gastric acid)), thereby transitioning sensor 250 from the compressed configuration to the expanded configuration, and increasing a closest distance between the first and the second electrode surfaces from the closest compressed-configuration distance D3 to the closest expanded-configuration distance D4.

For some applications, first and second electrodes 251 and 253 further comprise first and second elongate support structures 262 and 264, respectively.

First elongate support structure 262 is coupled to circuitry 256 at a first-structure coupling site 263 along first elongate support structure 262. First electrode surface 252 is (A) electrically coupled to circuitry 256 via first elongate support structure 262, and (B) disposed at a first electrode site 267 along first elongate support structure 262; when sensor 250 is in the expanded configuration, first electrode site 267 is disposed (x) within 2 mm of an end of first elongate support, structure 262, measured along first elongate support structure 262, and (y) at least 3 mm from first-structure coupling site 263, measured along first elongate support structure 262. For some applications, first electrode 151 comprises a partially insulated wire, an insulated portion of which serves as first elongate support structure 262, and a non-insulated portion, of which serves as first electrode surface 252.

Second elongate support structure 264 is coupled to circuitry 256 at a second-structure coupling site 265 along second elongate support structure 264, Second electrode surface 254 is (A) electrically coupled to circuitry 256 via second elongate support structure 264, and (B) disposed at a second electrode site 269 along second elongate support structure 264; when sensor 250 is in the expanded configuration, second electrode site 269 is disposed, (x) within 2 mm of an end of second elongate support structure 264, measured along second elongate support structure 264, and (y) at least 3 mm from second-structure coupling site 265, measured along second elongate support structure 264. For some applications, second electrode 153 comprises a partially insulated wire, an insulated portion of which serves as second elongate support structure 264, and a non-insulated portion of which serves as second electrode surface 254.

For some applications, sensor 250 comprises hydrogel 224, which, as mentioned above, is configured to undergo expansion upon contact with a liquid, thereby transitioning sensor 250 from the compressed configuration to the expanded configuration. First and second elongate support structures 262 and 264 are arranged such that the expansion of hydrogel 224 increases a closest distance between the first and the second electrode surfaces from closest compressed-configuration distance D3 to the closest expanded-configuration distance D4. For some applications, first and second elongate support structures 262 and 264 and circuitry 256 are embedded in hydrogel 224. For some applications, an expanded volume of hydrogel 224 equals at least 1.5 times a compressed volume of hydrogel 224, such as at least 3 times the compressed volume. For some applications, hydrogel 224 is generally spherical when sensor 250 is in both the compressed configuration and the expanded configuration.

In the applications described with reference to FIGS. 10A-B, first and second electrodes 251 and 253 (including first and second elongate support structures 262 and 264, if provided) are typically mechanically-passive, i.e., do not mechanically aid in the expansion of sensor 250 from the compressed configuration to the expanded configuration.

The detectable signal emitted by sensor 250 is detected by sensing unit 60, such as described hereinabove with reference to FIG. 5, mutatis mutandis.

Reference is now made to FIGS. 9A-B and 10A-B. In some applications of the present invention, apparatus is provided for use with an oral dosage form, the apparatus comprising a sensor, which is configured to assume compressed and expanded configurations, and which comprises (a) first and second electrodes, which comprise first and second electrode surfaces, respectively, and (b) circuitry, which is electrically coupled to the first and the second electrode surfaces. The sensor is:

configured such that when the sensor is in the compressed configuration, the first and the second electrode surfaces are disposed at a closest compressed-configuration distance from each other, and

configured such that when the sensor is in the expanded configuration, the first and the second electrode surfaces are disposed at a closest expanded-configuration distance from each other, the closest expanded-configuration distance equal to at least 2 times the closest compressed-configuration distance, and

configured to drive a current between the first and the second electrode surfaces when the sensor is in the expanded configuration.

For some applications, the oral dosage form is drug capsule, while for other applications, the oral dosage form is a drug tablet, such as a disk-shaped drug tablet or a caplet (an oval-shaped tablet in the general shape of a capsule). For some applications, the apparatus further comprises the oral dosage form.

For some applications, the sensor comprises a hydrogel that is configured to undergo expansion upon contact with a liquid (e.g., having a pH of 3 (such as gastric acid)), and to increase a closest distance between the first and the second electrode surfaces upon expansion. In other words, the first and the second electrode surfaces are disposed at a closest initial-configuration distance from each other, such as shown in FIGS. 9A and 10A, and a closest expanded-configuration distance that is greater than the closest initial-configuration distance, as shown in FIGS. 9B and 10B. For some applications, the closest expanded-configuration distance equals at least 1.5 times (e.g., 2 times) the closest initial-configuration distance.

For some applications, the hydrogel is provided as an element of ring 132, described hereinabove with reference to FIGS. 9A-B, or as an element of sensor 250, described hereinabove with reference to FIGS. 10A-B. Alternatively, the hydrogel is otherwise attached to the oral dosage form. For example, for applications in which the oral dosage form comprises a disk-shaped drug tablet, the hydrogel may be attached to one or both of the major surfaces of the disk-shaped drug tablet, e.g., the hydrogel itself may be disk-shaped.

In an application of the present invention, a sensing apparatus is provided for use with an oral dosage form containing an oral drug. The sensing apparatus comprises a piezoelectric crystal and a striking element. The striking element is initially restrained from contacting the piezoelectric crystal by a dissolvable element. The dissolvable element is configured to dissolve upon contact with a target physiological liquid inside a body of a human subject, such as gastric acid, after the oral dosage form has been swallowed with the sensing apparatus attached thereto. Upon release, the striking element strikes the piezoelectric crystal, thereby deforming the crystal. As a result of the deformation, the crystal generates a voltage pulse. The voltage pulse is detectable by a separate sensing unit, which is typically configured to be disposed external the subject's body. The energy used by the striking element to strike the crystal may be provided, for example, by a loaded spring, or by gas energy developed as a result of a local chemical reaction. This sensing apparatus may be used alone or in combination with any of the sensing apparatus described herein.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
an oral drug;
a drug capsule containing the oral drug; and
a sensing apparatus, which comprises:
a housing, which is shaped so as to define exactly one hemispherical portion and exactly one cylindrical portion, which together define an internal surface tightly fitted to at least a portion of an external surface of the drug capsule; and
a sensor, which comprises:
first and second electrodes, which comprise first and second electrode surfaces, respectively; and
circuitry, which (a) is attached to the housing, (b) is electrically coupled to the first and the second electrode surfaces, and (c) is configured to drive a current between the first and the second electrode surfaces.

2. The apparatus according to claim 1, wherein the at least a portion is less than the entire external surface of the drug capsule.

3. The apparatus according to claim 1, wherein the drug capsule comprises a capsule selected from the group consisting of: a hard-shelled capsule and a soft-shelled capsule.

4. The apparatus according to claim 1, wherein the at least a portion is the entire external surface of the drug capsule.

5. The apparatus according to claim 4, wherein the housing is a first housing, and wherein the sensing apparatus further comprises a second housing, which is sized and shaped to engage the first housing.

6. The apparatus according to claim 1, wherein the circuitry is attached inside the housing.

7. The apparatus according to claim 1, wherein the circuitry is attached outside the housing.

8. The apparatus according to claim 1, wherein a shortest path between the first and the second electrode surfaces that does not pass through any elements of the apparatus is at least 4 mm.

9. The apparatus according to claim 8, wherein the shortest path is no more than 20 mm.

10. The apparatus according to claim 1, wherein the circuitry is attached to the hemispherical portion of the housing.

11. The apparatus according to claim 1, wherein the first electrode surface is attached to the hemispherical portion.

12. The apparatus according to claim 1, wherein the second electrode surface is attached to the housing within 3 mm of a far end of the cylindrical portion from the hemispherical portion.

13. The apparatus according to claim 12, wherein the first electrode surface is attached to the hemispherical portion.

14. The apparatus according to claim 1, wherein the first electrode surface is disposed inside the housing, and the second electrode surface is disposed outside the housing.

15. The apparatus according to claim 1, wherein the housing comprises a material having an electrical resistance of at least 100 ohms.

16. The apparatus according to claim 1, wherein the housing comprises gelatin.

17. The apparatus according to claim 1, wherein the housing is non-biodegradable.

18. The apparatus according to claim 1, wherein the housing is configured, when submerged in a liquid having a pH of 3, to remain attached to the circuitry for at least one minute.

19. The apparatus according to claim 1, wherein the drug capsule is configured, when submerged in a liquid having a pH of 3, to dissolve to release the oral drug in a first amount of time, and wherein the housing is configured, when submerged in the liquid having the pH of 3, to remain attached to the circuitry for at least a second amount of time greater than the first amount of time.

20-41. (canceled)

42. A method comprising:
receiving, by a human subject, (a) a drug capsule containing an oral drug and (b) a sensing apparatus, which includes (i) a housing, which is shaped so as to define exactly one hemispherical portion and exactly one cylindrical portion, which together define an internal surface tightly fitted to at least a portion of an external surface of the drug capsule, and (ii) a sensor, which includes (A) first and second electrodes, which include first and second electrode surfaces, respectively, and (B) circuitry, which (a) is attached to the housing, (b) is electrically coupled to the first and the second electrode surfaces, and (c) is configured to drive a current between the first and the second electrode surfaces; and
swallowing, by the human subject, (a) the drug capsule and (b) the sensing apparatus while the internal surface is tightly fitted to the at least a portion of the external surface of the drug capsule.

43-59. (canceled)

60. A method of assembly comprising:
providing (a) a drug capsule containing an oral drug and (b) a sensing apparatus, which includes (i) a housing, which is shaped so as to define exactly one hemispherical portion and exactly one cylindrical portion, which together define an internal surface for tight fitting to at least a portion of an external surface of the drug capsule, and (ii) a sensor, which includes (A) first and second electrodes, which include first and second electrode surfaces, respectively, and (B) circuitry, which (a) is attached to the housing, (b) is electrically coupled to the first and the second electrode surfaces, and (c) is configured to drive a current between the first and the second electrode surfaces;
holding the drug capsule; and
attaching the sensing apparatus to the drug capsule by tightly fitting the internal surface to the at least a portion of the external surface of the drug capsule.

61. The method according to claim 60, wherein providing the drug capsule containing the oral drug comprises filling the drug capsule with the oral drug.

62-83. (canceled)
